(19) United States
(12) Patent Application Publication
(10) Pub. No.: US 2022/0090195 A1
(43) Pub. Date: Mar. 24, 2022

(54) STRUCTURE AND APPLICATION OF DOUBLE-STRANDED OLIGONUCLEOTIDE NUCLEIC ACID PROBE

(71) Applicant: ACADEMY OF MILITARY MEDICAL SCIENCES, Beijing 100039 (CN)

(72) Inventors: Shengqi WANG, Beijing (CN); Qiqi LIU, Beijing (CN); Liyan LIU, Beijing (CN); Ying ZHANG, Beijing (CN); Yi ZHAO, Beijing (CN)

(21) Appl. No.: 17/419,329

(22) PCT Filed: Mar. 12, 2019
(86) PCT No.: PCT/CN2019/077879

§ 371 Date: Jun. 29, 2021

(30) Foreign Application Priority Data
Dec. 29, 2018 (CN) 201811643407.0

(51) Int. Cl. C12Q 1/6876; C12Q 1/686; C12Q 1/6806

(52) U.S. Cl. CPC C12Q 1/6876; C12Q 1/686; C12Q 2563/107; C12Q 2533/107; C12Q 1/6806

(57) ABSTRACT

Disclosed by the present application are a structure of a double-stranded oligonucleotide nucleic acid probe, a method of use and applications thereof in nucleic acid fluorescence qualitative and quantitative analysis, medical diagnosis and life science researches. The double-stranded oligonucleotide nucleic acid probe is composed of two completely or partially base-complementary oligonucleotide strands; the end of each oligonucleotide strand may be connected to a fluorescent group or a corresponding fluorescent quenching group; and the two oligonucleotide probe strands may hybridize with a target nucleic acid sequence to be tested.

### CROSS-REFERENCE TO RELATED APPLICATION

The present disclosure claims the priority to the Chinese patent application filed on Dec. 29, 2018 with the Chinese Patent Office with the filing number 201811643407.0, and entitled “Structure and Application of Double-stranded Oligonucleotide Nucleic Acid Probe”, which is incorporated by reference herein in entirety.

### TECHNICAL FIELD

The present disclosure relates to a tool for biological detection technology, in particular, a double-stranded oligonucleotide nucleic acid probe, a method of using the same, and application of the same in gene fluorescence qualitative and quantitative analysis, medical diagnosis and other life science researches, belonging to the technical field of gene detection.

### BACKGROUND ART

Currently, the fluorescence probe method is usually used in molecular diagnosis of infectious diseases and the like, individualized companion diagnosis, and other disease diagnosis applications. The fluorescence probe method relies on fluorescent resonance energy transfer (FRET) to realize detection, including TaqMan probes, molecular beacons, Scorpion probes, and the like. The method only detects a specific amplification product, and therefore the specificity is strong. Currently, the most widely used is the TaqMan probe technology, and this technology mainly utilizes 5′ exonuclease activity of Taq enzyme. Firstly, a probe capable of hybridizing to a PCR product is synthesized, 5′ end of the probe labels a fluorescent molecule, 3′ end labels a corresponding fluorescent quenching molecule, and quenching molecule at 3′ end can absorb fluorescence emitted by the fluorescent molecule at 5′ end. Under normal conditions, the probe theoretically does not emit fluorescence, but when there is a PCR product in a solution, the probe is bound with the PCR product, to activate the 5′ end exonuclease activity of the Taq enzyme, and cleave the probe into a mononucleotide, at the same time, the fluorescent group labeled on the probe is free, as a result, fluorescence is emitted and the number of cleaved fluorescent molecules is proportional to the number of PCR products, therefore, the concentration of an initial template can be calculated from the fluorescence signal intensity in a PCR reaction liquid. TaqMan probe technology has many drawbacks despite its wide application.

### SUMMARY

The present disclosure aims at providing a new structure of double-stranded oligonucleotide nucleic acid probe, a method of using the same, and use of the same in gene fluorescence qualitative and quantitative analysis, medical diagnosis, life science researches and other fields, to solve at least one problem existing in the prior art.

The basic principle of the present disclosure is as follows.

As shown in the schematic diagram of qualitative and quantitative analysis of a double-stranded oligonucleotide nucleic acid probe in FIG. 1, two probes are synthesized first, the two probes are each labeled with a fluorescent group at 5′ end as a reporter molecule (F1/F2), the two probes are each labeled with a fluorescent quenching molecule (Q1/Q2) at 3′ end corresponding to the 5′ end, and the two probes are completely or partially base-complementary. When two probes are bound to each other, fluorescence emitted by the probes may be simultaneously absorbed by the quenching groups on the same chain and a complementary chain, for example, F1 may be simultaneously absorbed by Q1 and Q2, F2 may be simultaneously absorbed by Q2 and Q1, and no fluorescence is generated in the solution; when the two probes are separated, the probes are bound with a PCR product, 5′ end exonuclease activity of Taq enzyme cleaves the probe into mononucleotides, and F1 and F2 dissociate to emit fluorescence. The double-stranded probe has the advantages of more thorough quenching and lower fluorescence background. Based on this concept, the present disclosure synthesizes the double-stranded oligonucleotide nucleic acid probe, wherein when there is no template in a PCR amplification system, the two probes are complementarily bound, and no fluorescence is generated in the solution; when there is a template in the amplification system, the two probes both preferentially bind to the template at a higher temperature, so that the two probes are separated and produce fluorescence with fluorescence intensity proportional to a concentration of the template in the solution, thereby quantitative determination of the template may be carried out.

The objective of the present disclosure is achieved as follows: a double-stranded oligonucleotide nucleic acid probe, consisting of two completely or partially base-complementary oligonucleotide chains, is prepared, wherein for the two probes, an end of each oligonucleotide chain may be linked to a fluorescent group or a corresponding fluorescent quenching group, and the two oligonucleotide probe chains may be both hybridized and bound with a partial fragment of a target DNA or RNA nucleic acid sequence to be detected according to the base pairing principle. Each probe of the double-stranded probe independently consists of 6-50 oligonucleotides, and preferably, a long-strand probe in the double-stranded probe consists of 25-30 nucleotides, and a short-strand probe consists of 15-25 nucleotides. The number of fluorescent molecules and quenching molecules linked to the probe may be 1-5, generally being 1 in consideration of cost and synthesis convenience. The fluorescent group may be one or more selected from the group consisting of FAM, HEX, TET, ROX, CY3, CYS, VIC, JOE, SIMA, Alexa Fluor 488, TexasRed or Quasar 670, and the quenching group linked to the other end of this chain may be one or more selected from the group consisting of TAMRA, Dabcyl, BHQ-1, BHQ-2, BHQ-3, MGB or Eclipse.

The present disclosure discloses a method for using a double-stranded oligonucleotide nucleic acid probe in gene detection, which includes the following steps:

(1) preparing a double-stranded oligonucleotide nucleic acid probe;

(2) designing and synthesizing a pair of upstream and downstream primers according to a gene sequence to be detected, wherein Tm values of the primers are lower than a Tm value of a long-strand probe, the primers do not overlap with the probe and are adjacent to two ends of the probe, and are 1-150 nucleotides from the two ends of the probe;

(3) adding a template to a reaction mixture containing the probe, the primers, a PCR buffer solution, magnesium ions or manganese ions, dNTPs, and a Taq DNA polymerase to perform regular PCR, and amplification for 25-60 cycles, wherein the nucleic acid as a template preferably has a length of 60-500 bases, more preferably 70-150 bases, and fluorescence values are recorded in annealing or extension of each cycle;

(4) performing regression analysis on the number of cycles of threshold fluorescence with a logarithm of an initial concentration of the template, preparing a standard curve, and performing quantitative analysis on a concentration of the gene to be detected, wherein the threshold fluorescence refers to the fluorescence intensity of the gene to be detected that is 2 times a background fluorescence variation coefficient.

According to the above detection steps, the double-stranded oligonucleotide nucleic acid probe of the present disclosure can be widely applied to gene fluorescence qualitative and quantitative analysis, medical diagnosis and other gene detection fields, and in particular, it has greater advantages in the simultaneous detection and typing of multiple genes, and high-sensitivity detection of genes.

In the double-stranded oligonucleotide nucleic acid probe of the present disclosure, the double-stranded probes are fluorescent probes and quenching probes to each other, the fluorescent group and the quenching group are closer to each other, the quenching is more thorough, and the fluorescence background is greatly reduced; the double-stranded oligonucleotide nucleic acid probe does not completely rely on exonuclease activity, and may label two or more fluorescent molecules, and the two probes both bind to the template, thus improving the detection sensitivity. In summary, the double-stranded oligonucleotide nucleic acid probe technology has a greater promotion and application value.

Specific embodiments of the present disclosure are further described in detail with reference to the accompanying drawings.

### BRIEF DESCRIPTION OF DRAWINGS

FIG. 1a is a schematic diagram showing that when two oligonucleotide chains provided in the present disclosure have unequal lengths, a relatively short chain starts to be reversely complementary from a 5′ end of a relatively long chain, reverse complementary regions of a relatively short probe and a relatively long probe being both within the region of the relatively long probe;

FIG. 1b is a schematic diagram showing that when two oligonucleotide chains provided in the present disclosure have unequal lengths, a relatively short chain starts to be reversely complementary from 3′ end of a relatively long chain, reverse complementary regions of a relatively short probe and a relatively long probe being both within the region of the relatively long probe;

FIG. 1c is a schematic diagram showing that when two oligonucleotide chains provided in the present disclosure have unequal lengths, a relatively short chain starts to be reversely complementary from the middle of a relatively long chain, reverse complementary regions of a relatively short probe and a relatively long probe being both within the region of the relatively long probe;

FIG. 1d is a schematic diagram showing that when two oligonucleotide chains provided in the present disclosure have unequal lengths, a relatively short chain starts to be reversely complementary from a 5′ end of a relatively long chain, non-reverse complementary regions of the two oligonucleotide chains being both outside the 5′ end of the relatively long probe;

FIG. 1e is a schematic diagram showing that when two oligonucleotide chains provided in the present disclosure have unequal lengths, a relatively short chain starts to be reversely complementary from a 3′ end of a relatively long chain, non-reverse complementary regions of the two oligonucleotide chains being both outside the 3′ end of the relatively long probe;

FIG. 1f is a schematic diagram showing that when two oligonucleotide chains provided in the present disclosure have unequal lengths, two oligonucleotide chains have mutant bases that are incompletely reversely complementary;

FIG. 1g is a schematic diagram showing that when two oligonucleotide chains provided in the present disclosure have an equal length, two chains have mutant bases that are incompletely reversely complementary;

FIG. 2 is a specific analysis chart of probes provided in Example 1 of the present disclosure;

FIG. 3 is a background analysis chart of the probes provided in Example 1 of the present disclosure;

FIG. 4 is an analysis chart of a detection range of the probes provided in Example 1 of the present disclosure;

FIG. 5 is a detection chart of a double-stranded oligonucleotide nucleic acid probe provided in Example 1 of the present disclosure for 10 IU/mL HBV nucleic acid quantitative detection standard substance;

FIG. 6 is a detection chart of a double-stranded oligonucleotide nucleic acid probe provided in Example 1 of the present disclosure for 10 IU/mL HBV nucleic acid quantitative detection standard substance;

FIG. 7 is a detection chart of a Taqman probe provided in Example 1 of the present disclosure for 10 IU/mL HBV nucleic acid quantitative detection standard substance;

FIG. 8 is a detection chart of the double-stranded oligonucleotide nucleic acid probe provided in Example 1 of the present disclosure for samples;

FIG. 9 is a detection chart of the Taqman probe provided in Example 1 of the present disclosure for samples;

FIG. 10 is a chart showing results of detection of 2C19*2 gene mutant type A/A by a double-stranded oligonucleotide probe provided in Example 2 of the present disclosure;

FIG. 11 is a chart showing results of detection of 2C19*2 gene mutant type G/G by the double-stranded oligonucleotide probe provided in Example 2 of the present disclosure; and

FIG. 12 is a chart showing results of detection of 2C19*2 gene mutant type G/A by the double-stranded oligonucleotide probe provided in Example 2 of the present disclosure.

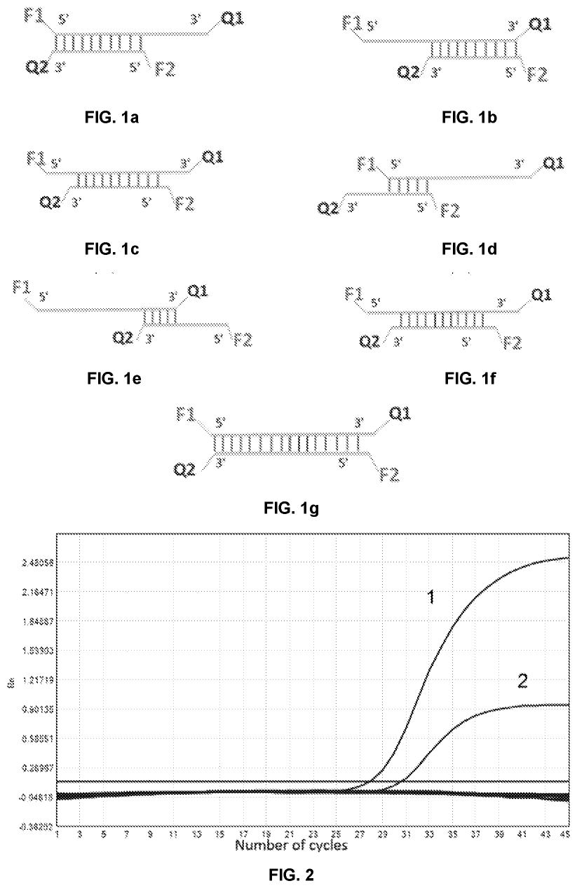

### DETAILED DESCRIPTION OF EMBODIMENTS

In order to further illustrate the technique and use of the double-stranded oligonucleotide nucleic acid probe, description is made with reference to the following examples, and the following examples are intended to illustrate rather than limit the present disclosure in any way.

### EXAMPLE 1

### Detection of Hepatitis B Virus by Double-Stranded Oligonucleotide Nucleic Acid Probe

1. Design of Primers and Probes for HBV Detection

According to the qualitative and quantitative analysis principle of double-stranded oligonucleotide nucleic acid probe, and in accordance with DNA sequence of a target molecule HBV to be detected, primers F and R, a long-strand oligonucleotide probe P1, a short-strand oligonucleotide probe P2, a Taqman probe P3, and a mutant base-containing oligonucleotide probe P4 were designed and synthesized. See Table 1 for the primer and probe sequences.

| Serial | | | | |
|---|---|---|---|---|
| No. | Name | Sequence | Length | Site |
| 1 | F | GYTATCGCTGGATGTGTCTGC | 21 | 366- |
| | | | | 386 |
| 2 | R | GACAAACGGGCAACATACCTT | 21 | 456- |
| | | | | 476 |
| 3 | P1 | FAM- | 27 | 403- |
| | | CCTCTKCATCCTGCTGCTATGCCTCAT- | | 429 |
| | | BHQ1 | | |
| 4 | P2 | FAM-GCATAGCAGCAGGATGM-BHQ1 | 17 | 408- |
| | | | | 424 |
| 5 | P3 | FAM- | 25 | 397- |
| | | CATATTCCTCTTCATCCTGCTGCTA- | | 421 |
| | | BHQ1 | | |
| 6 | P4 | FAM- | 27 | 403- |
| | | ATGAGGCATAGGTCGAGGATGMAGAGG- | | 429 |
| | | BHQ1 | | |

(1) Primers

The upstream primer F, having 21 nucleotides in total, was 17 nucleotides away from the long-strand oligonucleotide probe, and 22 nucleotides away from the short-strand oligonucleotide probe.

The downstream primer R, having 21 nucleotides in total, was 27 nucleotides away from the long-strand oligonucleotide probe, and 32 nucleotides away from the short-strand oligonucleotide probe.

(2) Probes

The long-strand oligonucleotide probe P1 was complementary to a minus strand of the target sequence, and consisted of 27 nucleotides, in which a 5′ end had a fluorescein molecule, and a 3′ end had a quenching molecule; the short-strand oligonucleotide probe P2 was complementary to a plus strand of the target sequence, and consisted of 17 nucleotides, in which a 5′ end had a fluorescein molecule, a 3′ end had a quenching molecule, the short-strand oligonucleotide probe P2 was 5 bases away from the probe P1 in position, and 10 bases shorter than the P1; the Taqman probe P3 was complementary to the minus strand of the target sequence, and consisted of 25 nucleotides, wherein a 5′ end had a fluorescein molecule, and a 3′ end had a quenching molecule; the mutant base-containing probe P4 had a mutant base which was incompletely reversely complementary to the plus strand of the target sequence; and the mutant base-containing probe P4 consisted of 27 nucleotides, wherein a 5′ end had a fluorescein molecule, and a 3′ end had a quenching molecule.

2. PCR detection

(1) PCR Detection by the Double-Stranded Oligonucleotide Nucleic Acid Probe

A PCR reaction system was formulated, including: 10× PCR buffer solution 4 μL, dNTPs 0.2 mmol/L, the upstream and downstream primers each 0.55 μmol/L, Taq DNA polymerase 2.5 U, a long-strand oligonucleotide probe 0.275 μmol/L, a short-strand oligonucleotide probe 0.330 μmol/L, and an HBV template 20 μL extracted with a nucleic acid extraction kit, a total reaction volume being 40 μL;

Reaction condition: 50° C., 2 min; 94° C., 2 min; 94° C., 15 s, 55 ° C., 45 s, 45 cycles in total, and collecting fluorescence during annealing.

(2) Fluorescent PCR Detection by the Double-Stranded Oligonucleotide Nucleic Acid Probe That Was Incompletely Reversely Complementary

A PCR reaction system was formulated, including: 10× PCR buffer solution 4 μL, dNTPs 0.2 mmol/L, the upstream and downstream primers each 0.55 μmol/L, Taq DNA polymerase 2.5 U, a long-strand oligonucleotide probe 0.275 μmol/L, an incompletely-reversely-complementary oligonucleotide probe 0.330 μmol/L, and an HBV template 20 μL extracted with a nucleic acid extraction kit, a total reaction volume being 40 μL;

Reaction condition: 50° C., 2 min; 94° C., 2 min; 94° C., 15 s, 55° C., 45 s, 45 cycles in total, and collecting fluorescence during annealing.

(3) Fluorescent PCR Detection by the Taqman Probe

A PCR reaction system was formulated, including: 10× PCR buffer solution 4 μL, dNTPs 0.2 mmol/L, the upstream and downstream primers each 0.55 μmol/L, Taq DNA polymerase 2.5 U, Taqman probe 0.275 μmol/L, and an HBV template 20 μL extracted with a nucleic acid extraction kit, a total reaction volume being 40 μL;

Reaction condition: 50° C., 2 min; 94° C., 2 min; 94° C., 15 s, 55° C., 45 s, 45 cycles in total, and collecting fluorescence during annealing.

3. Specificity of the Double-Stranded Oligonucleotide Nucleic Acid Probe

The primers were F and R, the probes were the double-stranded oligonucleotide nucleic acid probe (P1/P2) and the Taqman probe (P3), respectively, the templates were 10⁵ IU/mL hepatitis B virus (HBV), 10⁵ IU/mL hepatitis C virus (HCV), 10⁵ IU/mL hepatitis A virus (HAV), 10⁵ IU/mL human cytomegalovirus (CMV), 10⁵ IU/mL herpes simplex virus type I (HSV-1), and herpes simplex virus type II (HSV-2), respectively, and ddH₂O was a negative control. The PCR detection was performed according to operations in step 2.

Experiment results are as shown in FIG. 2. The double-stranded oligonucleotide nucleic acid probe and the Taqman probe respectively detected the 10⁵ IU/mL hepatitis B virus (HBV), 10⁵ IU/mL hepatitis C virus (HCV), 10⁵ IU/mL hepatitis A virus (HAV), 10⁵ PFU/mL human cytomegalovirus (CMV), 10⁵ PFU/mL herpes simplex virus type I (HSV-1), and 10⁵ PFU/mL herpes simplex virus type II (HSV-2), and the negative control ddH₂O. In the above, “S” type curve 1 is an amplification curve of the detection of the double-stranded oligonucleotide nucleic acid probe for 10⁵ IU/mL hepatitis B virus, “S” type curve 2 is an amplification curve of detection of the Taqman probe for 10⁵ IU/mL hepatitis B virus, and fluorescence intensities of the two both vary with the increase of the number of cycles; as for the two groups of probes for 10⁵ IU/mL hepatitis C virus (HCV), 10⁵ IU/mL hepatitis A virus (HAV), 10⁵ PFU/mL human cytomegalovirus (CMV), 10⁵ PFU/mL herpes simplex virus type I (HSV-1), 10⁵ PFU/mL herpes simplex virus type II (HSV-2), and the negative control ddH₂O, the fluorescence intensities of the two types of probes do not vary with the increase of the number of cycles, and results were all negative without amplification, being flat straight lines. It was thus demonstrated that the double-stranded oligonucleotide nucleic acid probe and the Taqman probe both have good specificity.

4. Quenching Efficiency of the Double-Stranded Oligonucleotide Nucleic Acid Probe

The primers were F and R, the probes were the double-stranded oligonucleotide nucleic acid probe (P1/P2) and Taqman probe (P3), respectively, and ddH₂O was a template. The amplification was performed for 15 cycles according to the operations in step 2, and the quenching efficiencies of the double-stranded oligonucleotide nucleic acid probe and the Taqman probe were detected.

As shown in FIG. 3, in the drawing, “1” group of flat straight lines with a fluorescence value of 900-1700 is for fluorescence signal background of amplification by the double-stranded oligonucleotide nucleic acid probe (P1/P2), and “2” group of flat straight lines with a fluorescence value of 3500-5300 is for fluorescence signal background of amplification by the Taqman probe. The fluorescence signal background of amplification by the Taqman probe is more than 3 times that by the double-stranded oligonucleotide nucleic acid probe. The results indicate that the double-stranded oligonucleotide nucleic acid probe is more thorough in quenching and renders lower background, and thus may solve the problem of influence of too high background on amplification.

5. Detection Range and Sensitivity of the Double-Stranded Oligonucleotide Nucleic Acid Probe (1) Linear Range Detection

The primers were F and R, the probes were the double-stranded oligonucleotide nucleic acid probe (P1/P2) and the Taqman probe (P3), respectively, and the template was an HBV nucleic acid quantitative detection standard substance at a concentration of 10⁹ IU/mL, and was diluted to 10⁹ IU/mL-10 IU/mL in 10-fold gradient. Amplification was performed according to the operations in step 2, to detect the quantitative range and sensitivity of the double-stranded oligonucleotide nucleic acid probe and the Taqman probe.

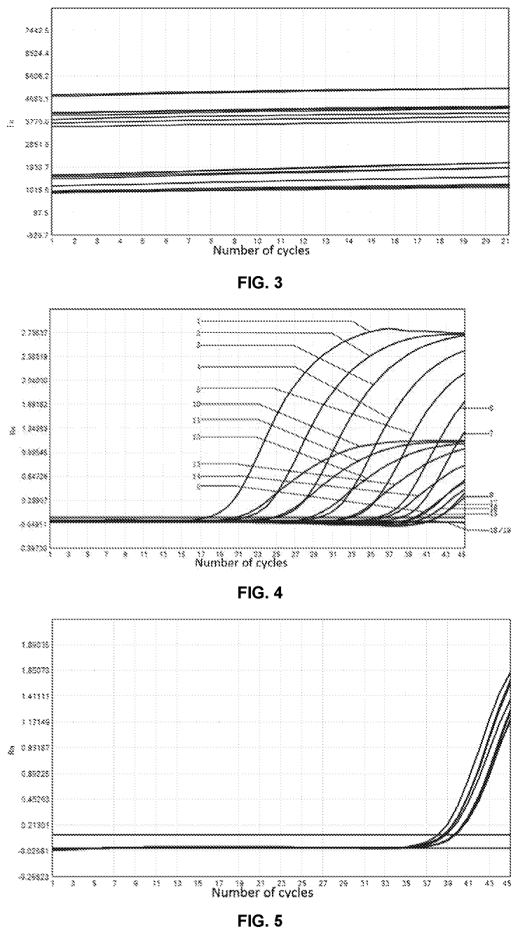

As shown in FIG. 4 and Table 2, when the template concentration is 10⁹ IU/mL-10 IU/mL, corresponding fluorescence responses could be seen from the group of double-stranded oligonucleotide nucleic acid probe (P1/P2) and the group of Taqman probe as the number of cycles changes. Curves 1-9 in the drawing are curves of amplification by the double-stranded oligonucleotide nucleic acid probe for 10⁹ IU/mL-10 IU/mL HBV nucleic acid quantitative detection standard substance, respectively, R²=0.9999, curves 10-17 are curves of amplification by the Taqman probe for 10⁹ IU/mL-10² IU/mL HBV nucleic acid quantitative detection standard substance, respectively, R²=0.9985, 18 is for 10 IU/mL HBV nucleic acid quantitative detection standard substance, 19 is for negative control, neither 18 nor 19 has amplification, and are flat straight lines. In the above, the fluorescence response intensity of the group of double-stranded oligonucleotide nucleic acid probe is significantly higher than that of the group of Taqman probe.

TABLE 2
Analysis Table of Probe Detection Range

| | Ct value | |
|---|---|---|
| | Double-stranded oligonucleotide | |
| Concentration (IU/mL) | nucleic acid probe (P1/P2) | Taqman probe |
| 10⁹ | 17.65 | 20.72 |
| 10⁸ | 21.09 | 24.71 |
| 10⁷ | 24.42 | 28.00 |
| 10⁶ | 28.50 | 32.46 |
| 10⁵ | 32.01 | 36.47 |
| 10⁴ | 35.76 | 39.59 |
| 10³ | 38.14 | 40.88 |
| 10² | 39.93 | 42.12 |
| 10 | 42.10 | No Ct |

(2) Sensitivity Detection

The primers were F and R, the probes were the double-stranded oligonucleotide nucleic acid probe (P1/P2), a double-stranded oligonucleotide nucleic acid probe (P1/P4), and the Taqman probe (P3), respectively, the template was an HBV nucleic acid quantitative detection standard substance at a concentration of 10 IU/mL. Detection was repeated 8 times. The amplification was performed according to the operations in step 2, to detect the lowest detection limits of the double-stranded oligonucleotide nucleic acid probe and the Taqman probe.

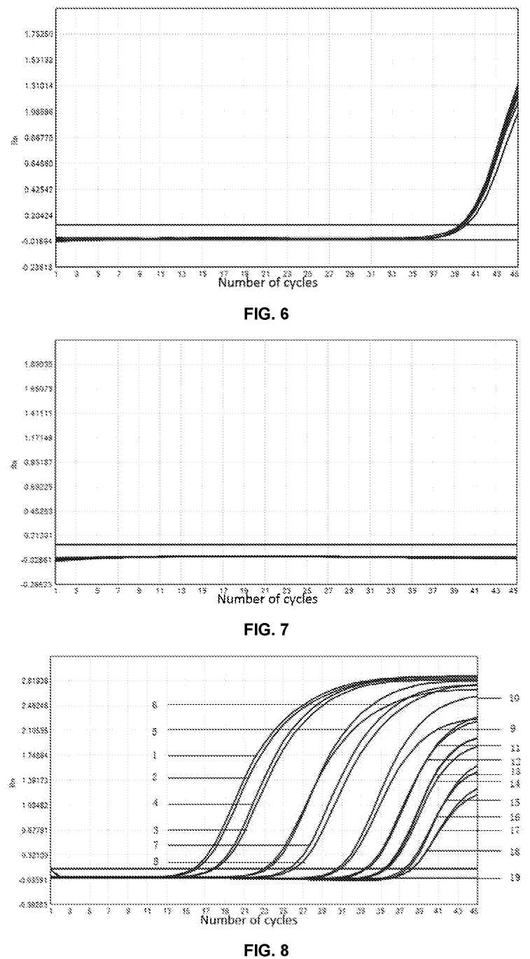

As shown in FIG. 5 (detection chart of the double-stranded oligonucleotide nucleic acid probe (P1/P2) for 10 IU/mL HBV nucleic acid quantitative detection standard substance), FIG. 6 (detection chart of the double-stranded oligonucleotide nucleic acid probe (P1/P4) for 10 IU/mL HBV nucleic acid quantitative detection standard substance), FIG. 7 (detection chart of the Taqman probe for 10 IU/mL HBV nucleic acid quantitative detection standard substance), and Table 3 and Table 4, when the template amount was 10 IU/mL, the group of double-stranded oligonucleotide nucleic acid probe (P1/P2) repeatedly performed detection 8 times, and there was fluorescence response 8 times, with typical “S” type curves; the group of double-stranded oligonucleotide nucleic acid probe (P1/P4) repeatedly detected 8 times, and there was fluorescence response 8 times, with typical “S” type curves; the group of Taqman probe repeatedly detected the 10 IU/mL HBV nucleic acid quantitative detection standard substance 8 times, none with fluorescence response. None of the above negatives was amplified, being flat straight lines.

TABLE 3
Detection Results of the Double-stranded Oligonucleotide Nucleic
Acid Probe (P1/P2) and the Taqman Probe for 10 IU/mL HBV Nucleic
Acid Quantitative Detection Standard Substance

| | Ct value | |
|---|---|---|
| | Double-stranded | |
| | oligonucleotide nucleic acid | |
| Concentration (10 IU/mL) | probe (P1/P2) | Taqman probe |
| well 1 | 38.60 | No Ct |
| well 2 | 39.67 | No Ct |
| well 3 | 39.64 | No Ct |
| well 4 | 39.61 | No Ct |
| well 5 | 38.59 | No Ct |
| well 6 | 38.08 | No Ct |
| well 7 | 39.76 | No Ct |
| well 8 | 38.87 | No Ct |

TABLE 4
Detection Results of the Double-stranded Oligonucleotide Nucleic
Acid Probe (P1/P4) and the Taqman Probe for 10 IU/mL HBV Nucleic
Acid Quantitative Detection Standard Substance

| | Ct value | |
|---|---|---|
| | Double-stranded | |
| | oligonucleotide nucleic acid | |
| Concentration (10 IU/mL) | probe (P1/P4) | Taqman probe |
| well 1 | 39.97 | No Ct |
| well 2 | 38.89 | No Ct |
| well 3 | 40.03 | No Ct |
| well 4 | 39.84 | No Ct |
| well 5 | 39.85 | No Ct |
| well 6 | 38.79 | No Ct |
| well 7 | 38.23 | No Ct |
| well 8 | 39.10 | No Ct |

It thus indicates that the double-stranded oligonucleotide nucleic acid probe may perform an accurate quantitative detection on samples at a concentration in the range of 10⁹ IU/mL-10 IU/mL, and provide a reference to the detection results for samples at a concentration of 10 IU/mL. The Taqman probe may perform an accurate quantitative detection on samples at a concentration in the range of 10⁹ IU/mL-10² IU/mL.

6. Quantitative Detection Analysis for HBV Clinical Sample

The primers were F and R, the probes were the double-stranded oligonucleotide nucleic acid probe (P1/P2) and the Taqman probe (P3), respectively, and the templates were 18 cases of HBV DNA positive sera with a fixed value. Amplification was performed according to the operations in step 2.

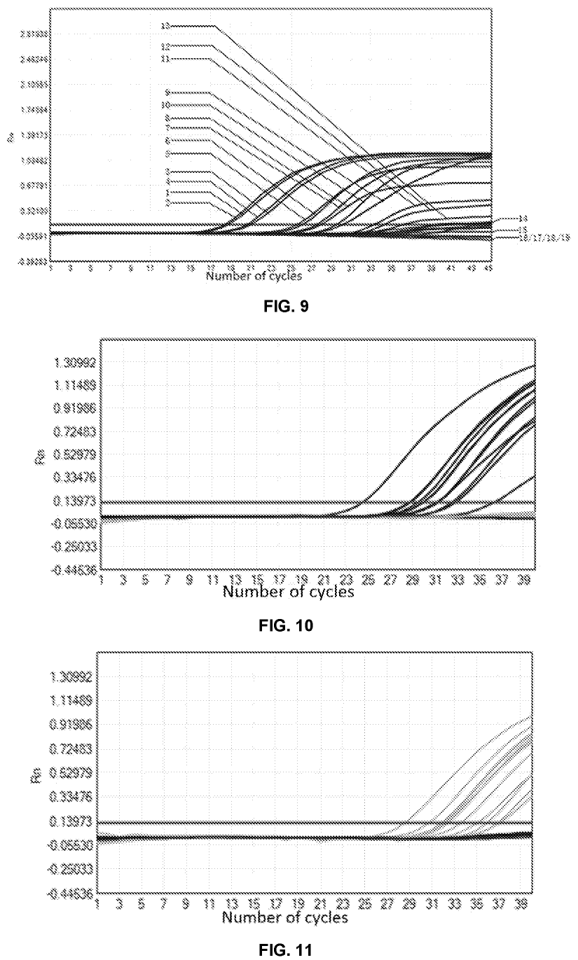

Results are as shown in FIG. 8 (sample detection chart of the double-stranded oligonucleotide nucleic acid probe), FIG. 9 (sample detection chart of the Taqman probe), and Table 5. From the results, it can be seen that within each concentration range, the double-stranded oligonucleotide nucleic acid probe can make effective detection, while the detection effect of the Taqman probe for the low-concentration samples is unfavorable. Results indicate that the double-stranded probe is capable of performing effective detection on the HBV clinical samples.

In the above, in FIG. 8, the double-stranded oligonucleotide nucleic acid probe detected 18 HBV clinical samples with a fixed value, and all the 18 samples could be detected, with typical “S” type curves. Sample concentrations of curves 1-2 were 6.31×10⁹ and 5.30×10⁹, sample concentrations of curves 3-4 were 1.65×10⁸ and 2.60×10⁸, sample concentrations of curves 5-6 were 2.77×10⁷ and 2.38×10⁷, sample concentrations of curves 7-8 were 1.50×10⁶ and 1.12×10⁶, sample concentrations of curves 9-10 were 1.50×10⁵ and 1.80×10⁵, sample concentrations of curves 11-12 were 3.17×10⁴ and 5.56×10⁴, sample concentrations of curves 13-14 were 5.26×10³ and 3.16×10³, sample concentrations of curves 15-16 were 3.14×10² and 1.95×10², sample concentrations of curves 17-18 were 7.10×10 and 4.90×10, and curve 19 was a negative, being a flat straight line, without amplification. In FIG. 9, the Taqman probe detected 18 HBV clinical samples with a fixed value, and 14 samples therein could be detected, with typical “S” type curves. 4 samples failed to be detected and were flat straight lines. Sample concentrations of curves 1-2 were 6.31×10⁹ and 5.30×10⁹, sample concentrations of curves 3-4 were 1.65×10⁸ and 2.60×10⁸, sample concentrations of curves 5-6 were 2.77×10⁷ and 2.38×10⁷, sample concentrations of curves 7-8 were 1.50×10⁶ and 1.12×10⁶, sample concentrations of curves 9-10 were 1.50×10⁵ and 1.80×10⁵, sample concentrations of curves 11-12 were 3.17×10⁴ and 5.56×10⁴, sample concentrations of curves 13-14 were 5.26×10³ and 3.16×10³, sample concentrations of curves 15-16 were 3.14×10² and 1.95×10², sample concentrations of curves 17-18 were 7.10×10 and 4.90×10, Curve 19 is a negative, being a flat straight line, without amplification.

TABLE 5
Table of Clinical Sample Detection Results of the Double-stranded
Oligonucleotide Nucleic Acid Probe and the Taqman Probe

| | | Ct value | |
|---|---|---|---|
| | Sample | Double-stranded | |
| Serial No. of | concentration | oligonucleotide | |
| sample | (IU/mL) | nucleic acid probe | Taqman probe |
| 1 | 6.31 × 10⁹ | 15.71 | 18.34 |
| 2 | 5.30 × 10⁹ | 16.07 | 18.77 |
| 3 | 1.65 × 10⁸ | 17.10 | 20.53 |
| 4 | 2.60 × 10⁸ | 18.04 | 20.98 |
| 5 | 2.77 × 10⁷ | 23.38 | 26.09 |
| 6 | 2.38 × 10⁷ | 23.05 | 25.54 |
| 7 | 1.50 × 10⁶ | 25.52 | 27.50 |
| 8 | 1.12 × 10⁶ | 26.10 | 28.10 |
| 9 | 1.50 × 10⁵ | 30.50 | 29.53 |
| 10 | 1.80 × 10⁵ | 30.94 | 30.18 |
| 11 | 3.17 × 10⁴ | 33.59 | 33.52 |
| 12 | 5.56 × 10⁴ | 33.35 | 34.34 |
| 13 | 5.26 × 10³ | 35.42 | 36.18 |
| 14 | 3.16 × 10³ | 35.59 | 37.50 |
| 15 | 3.14 × 10² | 37.73 | No Ct |
| 16 | 1.95 × 10² | 37.40 | No Ct |
| 17 | 7.10 × 10 | 38.22 | No Ct |
| 18 | 4.90 × 10 | 38.25 | No Ct |

### EXAMPLE 2

### Genotypic Detection of 2C19*2 (681 G>A) Mononucleotide Polymorphism Site of Double-Stranded Oligonucleotide Probe

1. Design of primers and probes for 2C19*2 gene detection

According to the qualitative and quantitative analysis principle of double-stranded oligonucleotide nucleic acid probe, and in accordance with DNA sequence of a target molecule 2C19*2 gene to be detected, upstream and downstream primers, a mutant chain oligonucleotide probe, and a wild chain oligonucleotide probe were designed and synthesized. See Table 6 for the primer and probe sequences.

| Serial | | |
|---|---|---|
| No. | Name | Sequence (5′-3′) |
| 1 | upstream | ATTATTGTTTTCTCTTAGATAT |
| | primer | |
| 2 | downstream | AAGTCCCGAGGGTTGTTGAT |
| | primer | |
| 3 | mutant | FAM-TATTTCCCAGGAACCCA-BHQ1 |
| | chain | |
| 4 | wild chain | HEX-TATGGGTTCCCGGGAAATAAT- |
| | | BHQ1 |

2. PCR Detection

(1) A PCR reaction system was formulated, including: 10×Buffer solution 2.5 μL, the upstream and downstream primers each 1.2 μL (10 μM), THE double-stranded oligonucleotide probe 0.6 μL (10 μM), Mg²+2.5 μL, 50×ST enzyme 0.5 μL (BIORI, China), and a human DNA template 3 μL extracted with a nucleic acid extraction kit, a total reaction volume being 25 μL.

Reaction condition: 50° C., 2 min; 95° C., 5 min; 95° C., 20 s, 60° C., 45 s, 40 cycles in total, and collecting fluorescence during annealing.

(2) Templates: 2C19*2 gene mutant type NA, wild type G/G, and heterozygous type G/A were each 12 cases, ddH₂O was a negative control. PCR detection was performed according to the operations in step (1).

3. Experiment Results

As shown in Table 6 and FIGS. 10-12:

TABLE 6
Table of Results of Detection of the Double-stranded Probe
for 36 2C19*2 Mutant, Wild, and Heterozygous Samples

| Serial No. of | | Ct value | |
|---|---|---|---|
| sample | Sample type | Mutant chain probe | Wild chain probe |
| 1 | mutant | 29.49 | No Ct |
| 2 | mutant | 32.53 | No Ct |
| 3 | mutant | 36.32 | No Ct |
| 4 | mutant | 31.11 | No Ct |
| 5 | mutant | 24.50 | No Ct |
| 6 | mutant | 32.80 | No Ct |
| 7 | mutant | 28.68 | No Ct |
| 8 | mutant | 28.83 | No Ct |
| 9 | mutant | 29.97 | No Ct |
| 10 | mutant | 30.01 | No Ct |
| 11 | mutant | 31.19 | No Ct |
| 12 | mutant | 31.23 | No Ct |
| 13 | wild | No Ct | 31.90 |
| 14 | wild | No Ct | 32.45 |
| 15 | wild | No Ct | 36.97 |
| 16 | wild | No Ct | 30.77 |
| 17 | wild | No Ct | 32.10 |
| 18 | wild | No Ct | 36.04 |
| 19 | wild | No Ct | 35.12 |
| 20 | wild | No Ct | 37.14 |
| 21 | wild | No Ct | 28.64 |
| 22 | wild | No Ct | 32.21 |
| 23 | wild | No Ct | 32.33 |
| 24 | wild | No Ct | 33.63 |
| 25 | heterozygous | 30.66 | 32.44 |
| 26 | heterozygous | 32.59 | 34.31 |
| 27 | heterozygous | 30.65 | 32.66 |
| 28 | heterozygous | 36.08 | 37.66 |
| 29 | heterozygous | 32.94 | 34.07 |
| 30 | heterozygous | 28.45 | 29.14 |
| 31 | heterozygous | 27.61 | 29.09 |
| 32 | heterozygous | 30.29 | 31.78 |
| 33 | heterozygous | 26.95 | 28.48 |
| 34 | heterozygous | 29.74 | 31.46 |
| 35 | heterozygous | 34.92 | 36.60 |
| 36 | heterozygous | 31.50 | 32.27 |

FIG. 10 shows the results of detection on the mutant type NA samples, wherein black lines represent the mutant chain probe labeled with an FAM fluorescent group, for detecting mutant sites, and except for the negative, the intensities of fluorescence signals increase with the increase of the number of cycles; grey lines represent the wild chain probe labeled with an HEX fluorescent group, for detecting wild sites, and no amplification is detected.

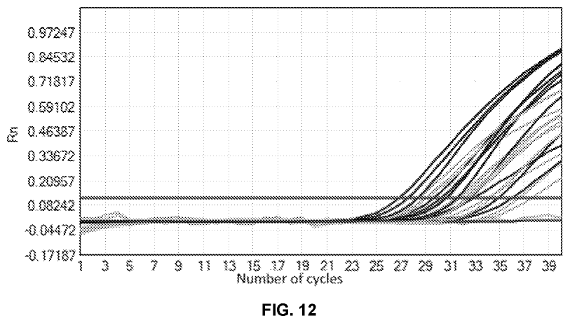

FIG. 11 shows the results of detection on the wild type G/G samples, wherein black lines represent the mutant chain probe labeled with an FAM fluorescent group, for detecting mutant sites, and no amplification is detected; grey lines represent the wild chain probe labeled with an HEX fluorescent group, for detecting wild sites, and except for the negative, the intensities of fluorescence signals increase with the increase of the number of cycles. FIG. 12 shows the results of detection on the heterozygous type G/A samples, wherein black lines represent the mutant chain probe labeled with an FAM fluorescent group, for detecting mutant sites; grey lines represent the wild chain probe labeled with an HEX fluorescent group, for detecting wild sites, and except for the negative, the intensities of two fluorescence signals both increase with the increase of the number of cycles.

From the above results, it can be seen that the double-stranded probe can detect genotype of mononucleotide polymorphism sites, and accurately detect the 2C19*2 gene mutant type NA, wild type G/G, heterozygous type G/A, and negative samples. For the mutant type samples, the mutant chain probe labeled with the FAM fluorescent group increases in the fluorescence signal intensity with the increase of the number of cycles, and the wild chain probe has no change; for the wild type samples, the wild chain probe labeled with the HEX fluorescent group increases in the fluorescence signal intensity with the increase of the number of cycles, and the mutant chain probe has no change; and for the heterozygous type samples, the fluorescence signal intensity of both increases with the increase of the number of cycles, and there is no change in the negatives.

### INDUSTRIAL APPLICABILITY

The double-stranded oligonucleotide nucleic acid probe provided in the present disclosure renders more thorough fluorescence quenching, greatly reduces the fluorescence background, and does not completely rely on exonuclease activity; besides, the double-stranded oligonucleotide nucleic acid probe may label two or more fluorescent molecules, thus improving the detection sensitivity and having a greater promotion and application value.

1. A double-stranded oligonucleotide nucleic acid probe structure, consisting of two completely or partially base-complementary oligonucleotide chains, wherein two probes each independently consist of 6-50 oligonucleotides, an end of each oligonucleotide chain is able to be linked to a fluorescent group or a corresponding fluorescent quenching group, and two oligonucleotide probe chains are both able to be hybridized and bound with a partial fragment of a target DNA or RNA nucleic acid sequence to be detected according to a base pairing principle.

2. The double-stranded oligonucleotide nucleic acid probe structure according to claim 1, wherein the fluorescent group or the fluorescent quenching group is linked at a 5′ end or a 3′ end of each oligonucleotide chain, respectively, and when a 5′ end or a 3′ end of one oligonucleotide chain is labelled with the fluorescent group, a 3′ end or a 5′ end of the other corresponding oligonucleotide chain bound thereto is labelled with the corresponding fluorescent quenching group.

3. The double-stranded oligonucleotide nucleic acid probe structure according to claim 1, wherein two ends of one oligonucleotide chain of the double-stranded oligonucleotide nucleic acid probe are both labelled with the fluorescent group, and two ends of the other oligonucleotide chain are both labelled with the fluorescent quenching group.

4. The double-stranded oligonucleotide nucleic acid probe structure according to claim 1, wherein the fluorescent group is one or more selected from the group consisting of FAM, HEX, TET, ROX, CY3, CY5, VIC, JOE, SIMA Alexa Fluor 488, TexasRed and Quasar 670.

5. The double-stranded oligonucleotide nucleic acid probe structure according to claim 1, wherein the fluorescent quenching group is one or more selected from the group consisting of TAMRA, Dabcyl, BHQ-1, BHQ-2, BHQ-3, MGB and Eclipse.

6. The double-stranded oligonucleotide nucleic acid probe structure according to claim 1, wherein the number of fluorescent groups and fluorescent quenching groups linked to each oligonucleotide chain is one or more, respectively.

7. The double-stranded oligonucleotide nucleic acid probe structure according to claim 1, wherein the two oligonucleotide chains have an equal length or unequal lengths.

8. The double-stranded oligonucleotide nucleic acid probe structure according to claim 7, wherein when the two oligonucleotide chains have unequal lengths, a relatively short oligonucleotide chain starts to be reversely complementary from a 5′ end, a 3′ end, and a middle of a relatively long oligonucleotide chain, reverse complementary regions of the relatively short oligonucleotide chain and the relatively long oligonucleotide chain are both in a region of the relatively long oligonucleotide chain, or non-reverse complementary regions of the two oligonucleotide chains are outside the 5′ end or the 3′ end of the relatively long oligonucleotide chain.

9. The double-stranded oligonucleotide nucleic acid probe structure according to claim 8, wherein a length of reverse complementation of the two oligonucleotide chains is generally 8-35 nucleotides.

10. The double-stranded oligonucleotide nucleic acid probe structure according to claim 8, wherein the relatively long oligonucleotide chain consists of 25-30 nucleotides, and the relatively short oligonucleotide chain consists of 15-25 nucleotides.

11. The double-stranded oligonucleotide nucleic acid probe structure according to claim 8, wherein the two oligonucleotide chains have mutant bases that are incompletely reversely complementary.

12. The double-stranded oligonucleotide nucleic acid probe structure according to claim 11, wherein the mutant bases are in number of 1-10.

13. The double-stranded oligonucleotide nucleic acid probe structure according to claim 7, wherein when the two oligonucleotide chains have an equal length, the two oligonucleotide chains have mutant bases that are incompletely reversely complementary.

14. The double-stranded oligonucleotide nucleic acid probe structure according to claim 13, wherein the mutant bases are in number of 1-10.

15. Use of the double-stranded oligonucleotide nucleic acid probe structure according to claim 1 in nucleic acid detection techniques.

16. The use according to claim 15, wherein the nucleic acid detection techniques comprise one or more selected from the group consisting of real-time fluorescent PCR, gene chip and membrane hybridization.

17. (canceled)

18. (canceled)

19. Method for using the double-stranded oligonucleotide nucleic acid probe structure according to claim 1 in nucleic acid detection techniques comprising real-time fluorescent PCR or reverse transcription real-time fluorescent PCR, comprising steps of:
(1) designing and preparing a double-stranded oligonucleotide nucleic acid probe and corresponding upstream and downstream amplification primers according to a target DNA or RNA nucleic acid sequence to be detected;
(2) formulating a PCR amplification reaction solution in a centrifuge tube, comprising the primers, the double-stranded oligonucleotide nucleic acid probe, a template, a PCR buffer system, magnesium ions or manganese ions, dNTPs and a Taq DNA polymerase;
(3) disposing the centrifuge tube containing the PCR reaction solution on a thermal cycler, to perform real-time fluorescent PCR amplification reaction, for 25-50 cycles, and recording fluorescence values during annealing or extension of each cycle; and
(4) performing regression analysis on a cycle number of threshold fluorescence with a logarithm of an initial concentration of the template, preparing a standard curve, and performing qualitative or quantitative analysis on the DNA or RNA to be detected.

20. The double-stranded oligonucleotide nucleic acid probe structure according to claim 2, wherein the fluorescent group is one or more selected from the group consisting of FAM, HEX, TET, ROX, CY3, CYS, VIC, JOE, SIMA, Alexa Fluor 488, TexasRed and Quasar 670.

21. The double-stranded oligonucleotide nucleic acid probe structure according to claim 2, wherein the fluorescent quenching group is one or more selected from the group consisting of TAMRA, Dabcyl, BHQ-1, BHQ-2, BHQ-3, MGB and Eclipse.

22. The double-stranded oligonucleotide nucleic acid probe structure according to claim 2, wherein the number of fluorescent groups and fluorescent quenching groups linked to each oligonucleotide chain is one or more, respectively.


SEQUENCE LISTING (10 sequences)

SEQ ID NO: 1 - DNA, 21 bp, Artificial sequence
  Upstream primer F
gytatcgctggatgtgtctgc

SEQ ID NO: 2 - DNA, 21 bp, Artificial sequence
  Downstream primer R
gacaaacgggcaacatacctt

SEQ ID NO: 3 - DNA, 27 bp, Artificial sequence
  Long-strand oligonucleotide probe P1
cctctkcatcctgctgctatgcctcat

SEQ ID NO: 4 - DNA, 17 bp, Artificial sequence
  Short-strand oligonucleotide probe P2
gcatagcagcaggatgm

SEQ ID NO: 5 - DNA, 25 bp, Artificial sequence
  Taqman probe P3
catattcctcttcatcctgctgcta

SEQ ID NO: 6 - DNA, 27 bp, Artificial sequence
  Mutant base-containing probe P4
atgaggcataggtcgaggatgmagagg

SEQ ID NO: 7 - DNA, 22 bp, Artificial sequence
  Upstream primer
attattgttttctcttagatat

SEQ ID NO: 8 - DNA, 20 bp, Artificial sequence
  Downstream primer
aagtcccgagggttgttgat

SEQ ID NO: 9 - DNA, 17 bp, Artificial sequence
  Mutant chain oligonucleotide probe
tatttcccaggaaccca

SEQ ID NO: 10 - DNA, 21 bp, Artificial sequence
  Wild chain oligonucleotide probe
tatgggttcccgggaaataat